(19) United States
(12) Patent Application Publication
(10) Pub. No.: US 2014/0206025 A1
(43) Pub. Date: Jul. 24, 2014

(54) Use Of Detection Of Aspartate Transaminase And Lactate Dehydrogenase In Early Evaluation Of Clinical Efficacy Of Antitumor Intervention Measure

(71) Applicants: Shifang Yang, Beijing (CN); Junsheng Cui, Beijing (CN); Peng Wei, Beijing (CN); Xiangjun Zheng, Beijing (CN); Bing Zhu, Beijing (CN)

(72) Inventors: Shifang Yang, Beijing (CN); Junsheng Cui, Beijing (CN); Peng Wei, Beijing (CN); Xiangjun Zheng, Beijing (CN); Bing Zhu, Beijing (CN)

(73) Assignee: Beijing Sunbio Biotech Co., Ltd., Beijing (CN)

(21) Appl. No.: 14/240,311

(22) PCT Filed: Aug. 30, 2011
(86) PCT No.: PCT/CN2011/001464

§ 371 Date: Apr. 3, 2014

(51) Int. Cl. G01N 33/50

(52) U.S. Cl. CPC G01N 33/5091
USPC 435/16; 435/26

(57) ABSTRACT

The present invention relates to a method for early evaluation of clinical efficacy of antitumor intervention measure, comprising evaluating the efficacy of the antitumor intervention measure by assaying whether the content of a tumor-damaging biomarker(s) in the blood of a patient having tumor rises as compared to the baseline level before treatment within a time window after the patients receives at least one antitumor intervention measure. In preferable embodiments, the tumor-damaging biomarker(s) is selected from a group consisting of Alanine Aminotransferase (ALT), Aspartate Transaminase (AST), Lactate Dehydrogenase (LDH); said tumor is multiple myeloma; said antitumor intervention measure is the administration of CPT alone or the administration of CPT in combination with thalidomide.

### TECHNICAL FIELD

The present invention relates to a method of early evaluation of efficacy of an antitumor intervention measure, specifically, it relates to a method of predicting the efficacy of the antitumor intervention measure by assaying the change of the level of a tumor-damaging biomarker(s) before and after the application of the antitumor intervention measure.

### BACKGROUND

As many as 7 million people all over the world die of cancer every year. The survival rate of the patients suffering from common malignant tumor has been very low. Especially those patients who have been diagnosed of suffering from cancer until a later stage have an even lower survival rate. For example, only less than 10% of the patients who are suffering from metastasis of colonic carcinoma and about 5% of the patients who are suffering from pancreatic carcinoma may survive 5 years or more. In fact, the “indiscrimination treatment” is still adopted for diagnosis and treatment of tumors currently. That is, identical method is used to all patients, i.e., diagnosis according to the types and stages of the tumor and application of the same treatment regardless of the biological characteristic of individual patient. The efficacy of each antitumor drug cannot achieve 100% and the efficacies of many antitumor drugs can not exceed 50%. It may be even lower in the patients suffering from relapse and refractory tumor. Thus, the majority of tumor patients are spending a lot of money, suffering enormous side-effects of the drugs, wasting valuable treatment opportunity and receiving ineffective treatment. Therefore, before or after receiving a certain intervention measure by a tumor patient, obtaining specific biological characteristics regarding the sensitivity of the patient towards such intervention measure would help the performance of personalized treatment on the tumor patient so as to dramatically improve the clinical benefit rate.

### SUMMARY OF THE INVENTION

The present invention discloses a method of predicting the efficacy of an antitumor intervention measure, which can predict the efficacy of the corresponding intervention measure earlier before the traditional and well recognized method can. The present invention is partly based on one or more measurable biomarker(s) released into the blood after the tumor cells are damaged by intervention measure. These biomarkers are mainly present intracellularly or on cytomembranes (including normal cells and tumor cells) under normal circumstances, and the baseline contents thereof in the blood circulation are relatively low. Increase of the content of one or more biomarker(s) in the serum predicts that the tumor cells are damaged by the intervention measure, thereby the efficacy of such intervention measure may be predicted.

The present invention provides a method of early evaluation of the efficacy of an antitumor intervention measure, comprising, before a tumor patient receives the antitumor intervention measure and after the patient receives at least one (i.e., at least once) antitumor intervention measure, assessing the efficacy of the antitumor intervention measure earlier by assaying whether the content of tumor-damaging biomarker(s) in the blood of the patient rises or not.

In a preferred embodiment, the method of the present invention predicts accurately the efficacy of CPT monotherapy and the combination of CPT with Thalidomide in the treatment of multiple myeloma by assaying the change of the content of Alanine Aminotransferase (ALT), Aspartate Transaminase (AST) and Lactate Dehydrogenase (LDH) after the treatment.

The present invention also provides the use of reagents assaying tumor-damaging biomarker in producing the reagent or kit for earlier evaluation of the clinical efficacy of the antitumor intervention measure, wherein said earlier evaluation of the clinical efficacy of the antitumor intervention measure comprises: evaluating the efficacy of the antitumor intervention measure by assaying whether the content of the tumor-damaging biomarker in the blood of a tumor patient rises as compared to the baseline level before treatment with the reagents assaying the tumor-damaging biomarker, within a time window after the tumor patient receives at least one antitumor intervention measure.

The present invention also provides a reagent or kit for earlier evaluation of the clinical efficacy of the antitumor intervention measure, comprising the reagents assaying the tumor-damaging biomarker.

The biomarker(s) released into the blood after the tumor cells are damaged in the present invention, which is abbreviated as “tumor-damaging biomarker(s)” below, includes but not limited to the following: Alanine Aminotransferase (ALT), Aspartate Transaminase (AST), Lactate Dehydrogenase (LDH), Uric acid, Creatinine, Heat Shock Protein 70 (HSP70), Heat Shock Protein 90 (HSP90), Monoclonal Immunoglobulin (or M protein), Immunoglobulin (IgG, IgA, IgD, IgM, IgE), Free Light Chain (FLC), β2 microglobulin (β2-MG) etc. Preferably, the tumor-damaging biomarker is selected from the group consisting of AST, ALT and LDH. In some embodiments, said “tumor-damaging biomarker” is AST, LDH, ALT, both AST and ALT, both AST and LDH, or all of AST, ALT and LDH. The tumor-damaging biomarker of the present invention comprises both the biomarkers specific to tumor cells, such as M protein of multiple myeloma, and the biomarkers not specific to tumor cells, such as Aspartate Transaminase (AST), Lactate Dehydrogenase (LDH). Thus, when predicting the efficacy of the antitumor intervention measure with the biomarkers not specific to tumor cells, it should be taken into consideration that the rise of content of such biomarkers may be resulted from the damage of non-tumor tissues. Therefore, the interference to the prediction may be reduced by excluding the damage of non-tumor tissues or improving the cutoff of the rise of the biomarker in view of specific clinical circumstances. In some embodiments, the rise of the tumor-damaging biomarker refers to 10% or more, for example, 20% or more, or 30% or more above the baseline value.

In the present invention, “time window” for assaying the content of the tumor-damaging biomarker refers to the time period before the earliest precise judgment achievable by conventional efficacy evaluation measures after the patient receives at least one (i.e., at least once) treatment of antitumor intervention measure. It is preferable to assay the tumor-damaging biomarker after one treatment, on the same day, the second or third day of the administration, for example, eight to forty-eight hours after receiving one antitumor treatment. If, the level of one or more tumor-damaging biomarker(s) relevant to the antitumor treatment rises in the blood of the patient, it predicts that the possibility that this treatment measure is effective to the patient increases greatly and the following multiple treatment periods may be continued. Contrarily, it shows that the possibility that this treatment measure is effective to the patient reduces greatly.

The effectiveness of a certain intervention measure in the present invention refers to that the patient may obtain a clinical response which is better than ‘stable disease’ in the conventional evaluation criterion for evaluating treatment efficacy on tumor, including but not limited to complete response, partial response, etc.

The tumor of the present invention refers to hematological tumor. According to the latest categorization criterion of the hematological tumors revised by WHO, the hematological tumors of the present invention include but not limited to chronic myeloproliferative disorders (CMPD), myelodysplastic/myeloproliferative disease (MDS/MPD), myelodysplastic syndrome (MDS), acute myeloid leukemia (AML), B cell tumor, T/NK cell tumor, hodgkin lymphoma, preferably B cell tumor. B cell tumor includes pre B cell tumor and mature B cell tumor, preferably mature B cell tumor. Mature B cell tumor includes chronic lymphocytic leukemia/small lymphocytic lymphoma, prolymphocytic leukemia, lymphoplasmacytic lymphoma/macroglobulinemia, splenic marginal zone B-cell lymphoma, splenic marginal zone B-cell lymphoma±villous lymphocyte, hairy cell leukemia B cell tumor, plasma cell myeloma/plasmacytoma, MALT extranodal (nodal) marginal zone B cell lymphoma, follicular lymphoma, mantle cell lymphoma, diffuse large B cell lymphoma, Burkitt's lymphoma, lymphomatoid granulomatosis, preferably plasma cell myeloma/plasmacytoma.

The antitumor intervention measure of the present invention refers to a medicament or treatment method having a killing effect on the tumor cell, including but not limited to the following measures: chemotherapy, radiotherapy, biologically targeted therapy, cellular immunotherapy, traditional Chinese medicine, etc. The above intervention measures may be used alone or in combination. The intervention measure which can induce the fast death of the tumor cell is preferred, such as cytotoxic drugs, apoptosis-inducing targeted drugs. The proapoptotic receptor agonists (PARAs) including recombinant human APO2L/TRAIL, the mutant of APO2L/TRAIL (e.g., recombinant mutant of human tumor necrosis factor-related apoptosis-inducing ligand, abbreviated as CPT) or agonistic monoclonal antibody is preferred. In addition, proteasome inhibitors such as Velcade, antibodies against CD20 such as rituximab are included.

The medicaments used in combination with PARAs medicaments include but not limited to paclitaxels, platinums, camptothecins, alkylating agents (e.g., Melphalan), antharcyclines (e.g., adriamycin), Thalidomide, Lenalidomide, proteasome inhibitors (e.g., Velcade), Glucocorticoid (e.g., Dexamethasone).

The method of the present invention is particularly suitable for targeted drug inducing apoptosis of the tumor cell, which acts exclusively on tumor cells, having relatively high specificity and less interference factor. After the death of a large amount of tumor cells, the contents are released into the blood and thus the content of the corresponding biomakers in the blood rises obviously.

The corresponding tumor-damaging biomarker for a specific tumor is known to those skilled in the art. Those skilled in the art can select suitable tumor-damaging biomarker and design corresponding indexes in view of the characteristics of the tumor.

The preferable tumor-damaging biomarker of the present invention includes but not limited to serum AST and/or LDH. The assay of the level of serum AST or ALT is generally used in the clinical evaluation of liver function. ALT is mainly distributed in liver, then skeletal muscle, kidney, cardiac muscle tissue etc. AST is mainly distributed in cardiac muscle, then liver, skeletal muscle and kidney tissue etc. Thus, AST and ALT is not specific for evaluating the abnormity of liver function. The injury of other organs, tissues or cells may also induce the abnormal change of the level of serum AST and/or ALT. For example, congestive heart failure, myocardiopathy and biliary obstruction etc. may elicit the rise of ALT; heart disease, myocardial infarction, acute pancreatitis, acute hemolytic anemia, severe burn, acute nephropathy, injury etc. may elicit the rise of AST. It was shown by previously published data that the content of AST and LDH is high in many types of human tumor tissues and human tumor cell lines whereas the content of ALT in tumor tissue or cell line is relatively low (http://www.proteinatlas.org). Thus, under the situation where other factors may be excluded (for example, drug induced liver injury, heart disease, myocardial infarction, acute pancreatitis, acute hemolytic anemia, severe burn, acute nephropathy, injury, etc), the sensitivity of the patient responding to antitumor treatment may be predicted earlier according to the rise of serum AST or LDH.

The AST of the present invention includes cytoplasmic form (c-AST) and mitochondrial form (m-AST). LDH includes five types, LDH-1, LDH-2, LDH-3, LDH-4 and LDH-5. The assay samples of ALT, AST, LDH etc. include serum and marrow. The assay method is not limited. Any quantitative assay method and apparatus is allowed. The conventional assay method includes enzyme-linked-ultraviolet continuous monitoring method, timing colorimetric method and enzyme-linked immunosorbent assay (ELISA), wherein the enzyme-linked-ultraviolet continuous monitoring method (ultraviolet spectrophotometry) is a dynamics method (rate assay) recommended by the International Federation of Clinical Chemistry and Laboratory Medicine (IFCC) and is widely used in the assay of ALT, AST and LDH since it is simple and fast. L-alanine and α-oxoglutarate may produce pyruvic acid and L-glutamate under the action of ALT. Pyruvic acid produces L-lactate under the action of lactic dehydrogenase. Meanwhile, NADH is oxidized to NAD+. The consumption amount of NADH may be monitored continuously at 340 nm so as to calculate the vigor of ALT. Similarly, AST is measured under the action of asparaginic acid and α-oxoglutarate, which produces oxaloacetic acid and L-glutamate using AST. Oxaloacetic acid produces malic acid under the action of malic dehydrogenase. Meanwhile, NADH is oxidized to NAD+. The reduction rate of absorbance is monitored continuously at 340 nm so as to calculate the concentration of the AST activity. LDH is measured by directly catalyzing oxidization of lactic acid to pyruvic acid using LDH. Meanwhile, NAD+ is reduced to NADH. The activity of LDH is obtained by assaying the increasing rate of absorbance at 340 nm wave length.

The reagent or kit for early evaluation of the clinical efficacy of the antitumor intervention measure may comprise reagents detecting one or more of AST, LDH and ALT. Preferably, the assay reagents are reagents which perform assay by enzyme-linked-ultraviolet continuous monitoring method, timing colorimetric method or enzyme-linked immunosorbent assay.

For example, the reagent or kit for early evaluation of the clinical efficacy of the antitumor intervention measure may comprise the following reagents:

(1) reagents for assaying AST, comprising asparaginic acid, α-oxoglutarate, malic dehydrogenase and NADH;

(1) reagents for assaying LDH, comprising lactic acid and NAD+;

(3) reagents for assaying ALT, comprising L-alanine, α-oxoglutarate, lactic dehydrogenase and NADH;

(4) reagents for assaying AST and ALT, comprising L-alanine, asparaginic acid, α-oxoglutarate, malic dehydrogenase, lactic dehydrogenase and NADH;

(5) reagents for assaying AST and LDH, comprising lactic acid, asparaginic acid, α-oxoglutarate, malic dehydrogenase, NAD+ and NADH;

(6) reagents for assaying ALT and LDH, comprising lactic acid, L-alanine, α-oxoglutarate, lactic dehydrogenase, NAD+ and NADH; or

(7) reagents for assaying AST, ALT and LDH, comprising lactic acid, L-alanine, asparaginic acid, α-oxoglutarate, lactic dehydrogenase, malic dehydrogenase, NAD+ and NADH.

The reagent or kit for early evaluation of the clinical efficacy of the antitumor intervention measure may optionally comprise other auxiliary reagents required for the detection, which are well-known to those skilled in the art, for example, buffers etc., and may be different according to different assay methods.

The present invention also relates to the use of reagents detecting one or more of AST, LDH and ALT in the preparation of a reagent or kit for early evaluation of the clinical efficacy of an antitumor intervention measure.

Recombinant mutant of human tumor necrosis factor-related apoptosis-inducing ligand (abbreviated as CPT) is a circularly permuted TRAIL (Tumor necrosis factor-related apoptosis-inducing ligand or APO2L) (Wiley, 1995; Pitti, 1996), belonging to antitumor medicaments of recombinant proteins. It was approved to be used in antitumor clinical trial by the China Food and Drug Administration in 2005. Currently, it was at phases II and III of the clinical trial. TRAIL/APO-2L acts on Death Receptor 4 and/or Death Receptor 5 on tumor cytomembrane in the form of homotrimer and selectively induces the apoptosis of multiple tumor cells, having no obvious toxic effect on normal cells (Cytokin & Growth Factor Reviews 14 (2003) 337-348). The mechanism of CPT is the same as that of TRAIL/APO2L, i.e., stimulating DR4/DR5 on tumor cytomembrane in the form of homotrimer and inducing the apoptosis of tumor cells. As compared to wild type TRAIL/APO-2L, CPT has a stronger antitumor activity (Acta Pharmacologica Sinica 26 (2005) 1373-1381). It was shown by the clinical trial in multiple myeloma (MM) that the objective response rate of CPT monotherapy in the patients who are suffering from relapsed and/or refractory multiple myeloma (RRMM) was about 30%, and there are still about 70% of patients who are not sensitive. Upon the analysis of clinical trial data, we found that the rise of AST and/or LDH in the serum of the patient after one or two CPT dosing and the treatment effect obtained by the subject were in good consistency. Thus, the clinical response of subjects to CPT may be predicted by the extent of the rise of AST and/or LDH in the serum after one or two CPT dosing.

The most common adverse event observed in CPT clinical trial is a mild or moderate acute liver damage. No other CPT induced damage such as heart, kidney, biliary obstruction etc. is observed (neither the rise of ALP, CK, creatine kinase, troponin associated with CPT nor the clinical representation of damages of these organs is observed). The liver damage induced by CPT may be represented as the rise of AST besides the rise of ALT (generally speaking, the extent of the rise of AST is slighter than that of ALT). Thus, in addition to CPT induced damage of bone marrow plasma cell tumor, CPT induced liver damage is a factor causing AST elevation in the treatment of MM by CPT. The interference of the AST rise induced by CPT-related liver damage is excluded by setting the ratio of the AST rise after the CPT dosing to a certain cutoff value and also setting the ratio of AST rise to ALT rise to a certain cutoff value, so as to earlier predict the response of the patient to CPT more accurately by the AST rise after CPT dosing. Similarly, the sensitivity of the patient responding to CPT is also predicted by setting the ratio of LDH rise after CPT dosing to a certain cutoff value alone or in combination with AST rise.

According to the requirement of analysis quality of Capability Demonstration Project of CL IA'88 of the USA, the allowable error range of the clinical tests of ALT, AST and LDH is target value±20%. According to “the recommended allowable error range for 11 conventional chemical test items in clinic” approved by the National Center for Clinical Laboratories, the allowable error range of the clinical tests of ALT, AST and LDH is target value±10%. Thus, according to the American criterion, the maximum ratio of the results of two tests of ALT, AST or LDH for a same sample is 1.50 (120%/80%). According to the Chinese criterion, the maximum ratio of the results of two tests of ALT, AST or LDH for a same sample is 1.22 (110%/90%). In other words, if the ratio of the results of ALT, AST or LDH obtained by two samplings is less than 1.22, such a rise may be caused by test error. The maximum ratio which is produced by testing the same sample twice and which might be related to the test error is different due to the possible difference of the allowable error ranges of these conventional test items in clinic in different countries or regions. Thus, the set value used to predict the efficacy by the rise of ALT, AST and LDH of the present invention is set according to the requirement of the allowable error ranges of these conventional test items in clinic in different countries or regions.

In addition to the test error of the test system, the factors influencing the accuracy for testing clinical samples include errors related to sample treatment, such as sampling manner, temperature and retention time, etc. As for ALT, AST and LDH, the result of such influence is rise. Thus, for improving the accuracy rate of predicting the efficacy of anti-tumor prevention measure, the set value of the rise of the tumor-damaging markers may also be set a little bit higher than the maximum ratio related to test error. However, if the set value is too high, the sensitivity of the prediction (the proportion of the predicted effective patients in the total effective patients) will reduce and the false negative rate (i.e., the effective patients is predicted to be ineffective patients) will increase. On the contrary, the specificity of the prediction (the proportion of the predicted ineffective patients in the total ineffective patients) will reduce and false positive rate (i.e., the ineffective patients are predicted to be effective patients) will increase. Thus, the setting of the set value also needs to take the preference of sensitivity and specificity into consideration. If the predicted specificity is expected to be high, the set value may be a little bit high. The present invention sets a set value to be 1.35 (ALT, AST) or 1.75 (LDH) when predicting the efficacy of CPT or the combined administration scheme comprising CPT by the rise of ALT, AST, LDH.

### DESCRIPTION OF THE DRAWINGS

FIG. 1 shows the change of the serum AST in RRMM patients at different times before or after CPT monotherapy.

FIG. 2 shows the change of the serum LDH in RRMM patients at different times before or after CPT monotherapy.

### EMBODIMENTS

The present invention will be further illustrated with the following Examples. These Examples are only described for the purpose of better explaining the present invention, without constituting a restriction to the scope claimed in any form.

### EXAMPLE 1

### The Rise of Serum AST and LDH Could Predict the Efficacy of CPT in Treating Patients with Multiple Myeloma

1. Inclusion Criteria

1.1 meeting the diagnostic criteria for MM, failed to treatment of first-line standard chemotherapy scheme or relapse/progress after relief;

1.2 age≧18

1.3 physical condition grade≧60

14 expected lifetime≧3 months

1.5 no chemotherapy, radiotherapy, targeted and anti-angiogenic drugs, interferon treatment and other research drugs was taken within two weeks;

1.6 no obvious dysfunction of main organs. The following laboratory indexes must meet the following requirements:

Blood: white blood cell≧2.0×10⁹/L, neutrophilic granulocyte≧1.0×10⁹/L, platelet count≧30×10⁹/L, hemoglobin≧60 g/L.

Liver function: serum total bilirubin, ALT and AST≦1.25 times of the upper limit of normal value.

Hepatitis B: positive for only surface antibody, core antibody or e antibody; positive for all of surface antigen, core antibody, e antibody but negative for HBV-DNA.

Hepatitis C: negative for HCV-RNA.

Renal function: creatinine clearance rate>10 ml/min.

Electrolyte: blood sodium and potassium must be within normal ranges.

1.7 Patients signed an informed consent upon understanding the details of the experiments.

2. Exclusion Criterion

2.1 gestational or lactating patients;

2.2 having a history of being allergic to biological products such as protein or allergic constitution;

2.3 having a history of viral hepatitis or other liver diseases, for example, liver cirrhosis, alcoholic liver disease, drug hepatitis, etc; positive for hepatitis B virus e antigen, surface antigen; positive for surface antigen, e antibody, core antibody and positive for HBV-DNA.

2.4 having mental diseases or a history of mental diseases;

2.5 having severe or uncontrollable diseases of important organs (heart and cerebral vessels, respiratory, digestion, nerve, etc) six months before enrollment, for example, myocardial infarction, III-IV stage of heart failure, angina pectoris, heart diseases with obvious clinical manifestation, left ventricular ejection fraction<0.5; severe conduction dysfunction; hypotension (Sitting systolic blood pressure≦90 mmHg and/or Sitting diastolic blood pressure≦60 mmHg);

2.6 suffering from other tumors;

2.7 those who were thought to be not suitable to be participated in this experiment;

3 Treatment Protocol

The patients received the CPT monotherapy after they were screened to be qualified for the experiment: 2.5 mg/kg of CPT was added to 250 ml of 5% glucose injection, and the intravenous infusion was continued for 1.5 hours±15 minutes (for those who were suffering from diabetes or having a history of diabetes, normal saline of 250 ml may be used instead), once a day for 14 consecutive days; each treatment observation period (treatment cycle) consisted of 21 days.

During the first treatment cycle, the patient was administrated with CPT for 14 days followed by a 7-day treatment-free period. Then, the second cycle began and the efficacy was evaluated after 14-day CPT treatment.

After two treatment cycles, CPT therapy could be continued for patients achieving a complete response, partial response or minimal response if it was regarded as beneficial by the investigators. Informed consent should be obtained. Treatment beyond 6 cycles was not permitted.

4. The Criteria for Efficacy Assessment of Multiple Myeloma

Clinical responses are evaluated according to EBMT (European Group for Blood and Marrow Transplantation) criteria.

4.1 complete response, CR

Meeting all of the following:

(1) Absence of the monoclonal protein (M protein) in serum and urine by immunofixation, maintained for a minimum of 6 weeks.

(2) <5% plasma cells in bone marrow smear and bone marrow biopsy (if biopsy is performed). If absence of monoclonal protein is sustained for 6 weeks it is not necessary to repeat the bone marrow test^{[2]}.

(3) No increase in size or number of lytic bone lesions (development of a compression fracture does not exclude response)^{[3]}.

(4) Disappearance of soft tissue plasmacytomas.

4.2 nearly complete response, nCR:

CR^{IF+}, absence of M protein in serum and urine (non-immunofixation electrophoresis assay).

4.3 partial response, PR:

Meeting all of the following:

(1) ≧50% reduction in the level of the serum monoclonal protein, maintained for a minimum of 6 weeks.

(2) Reduction in 24 h urinary light chain excretion either by ≧90% or to <200 mg, maintained for a minimum of 6 weeks.

(3) For patients with non-secretory myeloma only, ≧50% reduction in plasma cells in a bone marrow smear and bone marrow biopy, if biopsy is performed, maintained for a minimum of 6 weeks.

(4) ≧50% reduction in the size of soft tissue plasmacytomas (by radiography or clinical examination).

(5) No increase in size or number of lytic bone lesions (development of a compression fracture does not exclude response)^{[3]}.

4.4 minimal response, MR:

Meeting all of the following:

(1) 25-49% reduction in the level of the serum monoclonal protein maintained for a minimum of 6 weeks.

(2) 50-89% reduction in 24 h urinary light chain excretion, which still exceeds 200 mg/24 h, maintained for a minimum of 6 weeks.

(3) For patients with non-secretory myeloma only, 25-49% reduction in plasma cells in a bone marrow smear and bone marrow biopsy, if biopsy is performed, maintained for a minimum of 6 weeks.

(4) 25-49% reduction in the size of soft tissue plasmacytomas.

(5) No increase in the size or number of lytic bone lesions lesions (development of a compression fracture does not exclude response)^{[3]}.

4.5. no change, (NC):

Not meeting the criteria of either minimal response or progressive disease.

4.6 relapse from CR:

Meeting at least one of the following:

(1) Reappearance of serum or urinary monoclonal protein on immunofixation or routine electrophoresis, confirmed by at least one further investigation.

(2) >5% plasma cells in a bone marrow smear or bone marrow biopsy.

(3) Development of new lytic bone lesions or soft tissue plasmacytomas or definite increase in the size of residual bone lesions.

(4) Development of hypercalcaemia (corrected serum calcium>11.5 mg/dl or 2.8 mmol/l) not attributable to any other cause^{[6]}.

4.7 progressive disease (PD):

Meeting one or more of the following:

(1) >25% increase in the level of the serum monoclonal protein^{[4]}, which must also be an absolute increase of at least 5 g/l and confirmed by at least one repeated investigation.

(2) >25% increase in the 24 h urinary light chain excretion^{[4]}, which must also be an absolute increase of at least 200 mg/24 h and confirmed by at least one repeated investigation.

(3) >25% increase in plasma cells in a bone marrow smear or bone marrow biopsy^{[4]}, which must also be an absolute increase of at least 10%^{[4]}.

(4) Definite increase in the size of existing bone lesions or soft tissue plasmacytomas^{[4]}.

(5) Development of new bone lesions or soft tissue plasmacytomas.

(6) Development of hypercalcaemia (corrected serum calcium>11.5 mg/dl or 2.8 mmol/l) not attributable to any other cause^{[6]}.

Notes:

[1]. The criteria was revised from that reported by Blade et al.

[2]. If the absence of monoclonal protein maintained for 6 weeks, it was not necessary to repeat the bone marrow examination. The bone marrow examination was required for patients with hyposecretory or non-secretory myeloma (including 6-week follow-up examination).

[3]. Skeletal X-ray examination was not required for the definition of response, but if performed there must be no evidence of progression of bone disease (no increase in the size or number of lytic bone lesion).

[4] Calculation of any increase in the parameters shall be based on the minimum value unless regarded to be suspicious.

[5]. Increase of the size of the lesions referred to at least 50% increase of the product of maximum diameters.

[6]. Other clinical data might be required for the evaluation of the cause of the hypercalcemia before determining to be the reason of disease progression.

5. Efficacy Analysis

Of 37 subjects enrolled in the trial, 30 subjects received at least two cycles of CPT treatment and 7 subjects received one treatment cycle. The efficacy evaluation was shown in Table 1.

TABLE 1
Efficacy of CPT Monotherapy for the
treatment of Patients with RRMM

| Efficacy | Number of | Incidence |
|---|---|---|
| Evaluation | Subjects | Rate (%) |
| CR | 1 | 2.7 |
| PR | 12 | 32.4 |
| MR | 8 | 21.6 |
| NC | 10 | 27.0 |
| PD | 6 | 16.2 |

6. The Relationship Between the Change of Serum AST After CPT Treatment and Therapeutic Efficacy

After two days of CPT treatment and before administration on day 3, venous blood samples were collected and the serum AST and ALT were tested. It can be seen that the serum AST and ALT of the patients increased to varying degrees: some being the rise of AST alone; some being the rise of ALT alone; some being the rise of both AST and ALT (Table 2). ΔAST was used to refer to the degree of the AST elevation after CPT treatment (ΔAST=serum AST after treatment/baseline level). ΔALT was used to refer to the degree of ALT elevation after CPT treatment (ΔALT=serum ALT after treatment/baseline level). As the biomarkers of tumor-damage, ΔAST>1.35 and ΔAST/ΔALT>1.35 were regarded as positive in the present study, otherwise, negative. Thus, the interference of AST elevations caused by liver injuries could be excluded to a great degree. The response of the multiple myeloma patients to CPT was predicted by the change of ΔAST and ΔAST/ΔALT after CPT administration.

Table 2 showed the concentration of AST, ALT and LDH in the fasting venous blood samples collected from 37 patients after two dosing of CPT and before the third CPT dosing, and their clinical responses after having finished 1-2 cycles of treatment. Of 37 subjects, 18 was positive in the tumor-damaging biomarker (ΔAST>1.35 and ΔAST/ΔALT>1.35) (Table 3), with 11 achieved a PR (61.1%), 1 CR (5.6%), 3 MR (16.7%), 2 NC (11.1%), 1 PD (5.6%), and the clinical response rate (PR+CR) was 66.7%. Nineteen subjects was negative in the tumor-damaging biomarker (which could not meet the requirement of AST>1.35 and ΔAST/ΔALT>1.35) (Table 4), with 1 achieved a PR (5.3%), 5 MR (26.3%), 8 NC (42.1%), 5 PD (26.3%), and the clinical response rate (PR+CR) was 5.3% (no CR). In the patients whose tumor-damaging biomarker was positive, the percentage of PR+CR was 12.6 times of that of patients whose tumor-damaging biomarker was negative.

The sensitivity of this tumor-damaging biomarker in predicting the clinical response (PR+CR) of MM patients to CPT was 92.3% (12/13), and the specificity thereof was 75% (18/24). The predicted value of the positivity of the clinical response (PR+CR) was 66.7% (12/18) and the predicted value of the negativity of the clinical response (PR+CR) was 94.7% (18/19). A logistic regression analysis was conducted on the relevance of clinical response rate of MM patients to CPT with the rise of serum AST by SPSS statistics software, and the result showed that ΔAST was useful for predicting whether the patients would response to CPT treatment (P<0.005), with an odds ratio of 36.00 and 95% confidence interval of 3.84˜337.98 (Table 5).

7. The Relationship Between the Change of Serum LDH After CPT Treatment and the Efficacy

After two days of CPT treatment and before administration on day 3, venous blood samples of the patients were collected and the serum LDH levels were tested. It can be seen that the serum LDH content of the treated patients increased to varying degrees. ΔLDH was used to refer to the degree of the serum LDH elevation after CPT treatment (ΔLDH=serum LDH after treatment/baseline level). Nineteen subjects had a ΔLDH ratio (Table 2). ΔLDH≧1.75 was regarded as positive in tumor-damaging biomarker, otherwise, negative. The response of the multiple myeloma patients to CPT was predicted by the change of ΔLDH after CPT administration.

There were 7 patients with positive tumor-damaging biomarker, wherein 5 of them had a clinical response of PR or better (PR+CR) (71.4%). There were 12 patients with negative tumor-damaging biomarker, wherein 1 of them had a clinical response of PR or better (PR+CR) (8.3%). In the patients whose tumor-damaging biomarker was positive, the percentage of PR+CR was 8.6 times that of patients whose tumor-damaging biomarker was negative.

The sensitivity of the tumor-damaging biomarker ΔLDH in predicting the clinical response (PR+CR) of MM patients to CPT was 83.33% (5/6), and the specificity thereof was 84.62% (11/13). The predicted value of the positivity of the clinical response (PR+CR) was 71.43% (5/7) and the predicted value of the negativity of the clinical response (PR+CR) was 91.67% (11/12). A logistic regression analysis was conducted on the relevance of clinical response rate of MM patients to CPT with the rise of serum LDH by SPSS statistics software, and the result showed that [symbol] LDH was useful for predicting whether the patients would response to CPT treatment (P<0.05), with an odds ratio of 27.50 and 95% confidence interval of 2.0˜378.84 (Table 6).

TABLE 2
Changes of Serum AST, ALT and LDH and the Efficacy of RRMM
Patients after CPT Monotherapy

| | | | | | | Before | | | After two days | | |
|---|---|---|---|---|---|---|---|---|---|---|---|
| | | The fold increased after CPT treatment | | | | CPT treatment | | | of CPT treatment | | |
| No. | Efficacy | ΔAST | ΔALT | ΔLDH | ΔAST/ΔALT | AST | ALT | LDH | AST | ALT | LDH |
| 1 | PR | 24.50 | 11.33 | 5.73 | 2.16 | 6 | 6 | 120 | 147 | 68 | 688 |
| 2 | PR | 17.65 | 3.20 | 20.37 | 5.52 | 20 | 30 | 197 | 353 | 96 | 4012 |
| 3 | PR | 12.94 | 0.92 | | 14.12 | 18 | 12 | 154 | 233 | 11 | |
| 4 | PR | 12.50 | 1.13 | 4.82 | 11.11 | 17 | 8 | 114 | 24 | 9 | 549 |
| 5 | CR | 8.07 | 1.67 | 1.01 | 4.84 | 15 | 12 | 148 | 121 | 20 | 149 |
| 6 | PR | 7.75 | 1.05 | 14.60 | 7.38 | 16 | 20 | 84 | 124 | 21 | 1226 |
| 7 | PR | 7.29 | 4.09 | | 1.78 | 21 | 44 | 228 | 153 | 180 | |
| 8 | NC | 6.79 | 4.91 | | 1.38 | 23.2 | 17.6 | 169 | 157.5 | 86.4 | |
| 9 | PR | 6.52 | 0.99 | | 6.57 | 19.2 | 12.9 | 225.8 | 125.1 | 12.8 | |
| 10 | NC | 6.50 | 7.95 | | 0.82 | 28 | 21 | 168 | 182 | 167 | |
| 11 | PR | 6.11 | 1.40 | | 4.36 | 28 | 15 | 567 | 171 | 21 | |
| 12 | PR | 5.40 | 0.88 | 6.90 | 6.14 | 10 | 25 | 80 | 54 | 22 | 552 |
| 13 | PD | 4.32 | 3.27 | 4.75 | 1.32 | 31 | 15 | 297 | 134 | 49 | 1410 |
| 14 | PD | 3.50 | 2.67 | 1.26 | 1.31 | 8 | 21 | 110 | 28 | 56 | 139 |
| 15 | MR | 3.13 | 1.84 | | 1.70 | 30 | 25 | | 94 | 46 | |
| 16 | NC | 2.64 | 6.26 | 0.94 | 0.42 | 33 | 19 | 175 | 87 | 119 | 165 |
| 17 | PR | 2.29 | 1.65 | | 1.39 | 15.3 | 13.6 | 109.9 | 35 | 22.4 | |
| 18 | PD | 2.27 | 0.71 | | 3.21 | 11 | 24 | 161 | 25 | 17 | |
| 19 | NC | 2.11 | 2.20 | 2.00 | 0.96 | 9 | 5 | 166 | 19 | 11 | 332 |
| 20 | NC | 2.07 | 2.00 | 1.62 | 1.04 | 14 | 25 | 63 | 29 | 50 | 102 |
| 21 | MR | 1.96 | 1.39 | 1.72 | 1.41 | 27 | 28 | 367 | 53 | 39 | 633 |
| 22 | PR | 1.92 | 0.49 | | 3.88 | 37.6 | 57.9 | 91 | 72.1 | 28.6 | |
| 23 | MR | 1.79 | 2.00 | 1.28 | 0.89 | 14 | 11 | 130 | 25 | 22 | 166 |
| 24 | NC | 1.63 | 2.13 | | 0.77 | 51 | 64 | | 83 | 136 | |
| 25 | NC | 1.59 | 1.00 | | 1.59 | 22 | 14 | 139 | 35 | 14 | ND |
| 26 | MR | 1.38 | 0.95 | 0.94 | 1.44 | 8 | 22 | 127 | 11 | 21 | 120 |
| 27 | MR | 1.38 | 1.50 | 1.01 | 0.92 | 8 | 8 | 123 | 11 | 12 | 124 |
| 28 | PD | 1.36 | 1.45 | 0.94 | 0.93 | 7.8 | 6.6 | 97.7 | 10.6 | 9.6 | 91.9 |
| 29 | NC | 1.24 | 1.58 | | 0.78 | 21 | 24 | 208 | 26 | 38 | |
| 30 | MR | 1.15 | 0.72 | | 1.60 | 13 | 18 | 103 | 15 | 13 | ND |
| 31 | NC | 1.12 | 1.25 | 1.01 | 0.89 | 17 | 8 | 138 | 19 | 10 | 140 |
| 32 | MR | 1.11 | 1.05 | | 1.06 | 31.8 | 19.5 | 142 | 35.4 | 20.4 | |
| 33 | PD | 1.06 | 0.76 | 1.23 | 1.40 | 21.7 | 15.2 | 126.2 | 23 | 11.5 | 155.2 |
| 34 | MR | 1.06 | 1.03 | | 1.03 | 17 | 33 | 107 | 18 | 34 | |
| 35 | NC | 0.94 | 0.91 | | 1.04 | 34 | 11 | 100 | 32 | 10 | ND |
| 36 | PD | 0.89 | 0.45 | 0.84 | 1.98 | 18 | 29 | 92 | 16 | 13 | 77 |
| 37 | PR | 0.53 | 0.60 | | 0.88 | 24.7 | 20.5 | 109.2 | 13 | 12.2 | |

TABLE 3
RRMM Patients Positive in the Biomarker of AST Elevation
and the Efficacy after CPT Monotherapy*

| | | | | | | | After | |
|---|---|---|---|---|---|---|---|---|
| | | | | | Before | | two days | |
| | | The fold increased | | | CPT | | of CPT | |
| | Effic- | after CPT treatment | | | treatment | | treatment | |
| No | acy | ΔAST | ΔALT | ΔAST/ΔALT | AST | ALT | AST | ALT |
| 1 | PR | 24.50 | 11.33 | 2.16 | 6 | 6 | 147 | 68 |
| 2 | PR | 17.65 | 3.20 | 5.52 | 20 | 30 | 353 | 96 |
| 3 | PR | 12.94 | 0.92 | 14.12 | 18 | 12 | 233 | 11 |
| 4 | PR | 12.50 | 1.13 | 11.11 | 17 | 8 | 24 | 9 |
| 5 | CR | 8.07 | 1.67 | 4.84 | 15 | 12 | 121 | 20 |
| 6 | PR | 7.75 | 1.05 | 7.38 | 16 | 20 | 124 | 21 |
| 7 | PR | 7.29 | 4.09 | 1.78 | 21 | 44 | 153 | 180 |
| 8 | NC | 6.79 | 4.91 | 1.38 | 23.2 | 17.6 | 157.5 | 86.4 |
| 9 | PR | 6.52 | 0.99 | 6.57 | 19.2 | 12.9 | 125.1 | 12.8 |
| 11 | PR | 6.11 | 1.40 | 4.36 | 28 | 15 | 171 | 21 |
| 12 | PR | 5.40 | 0.88 | 6.14 | 10 | 25 | 54 | 22 |
| 15 | MR | 3.13 | 1.84 | 1.70 | 30 | 25 | 94 | 46 |
| 17 | PR | 2.29 | 1.65 | 1.39 | 15.3 | 13.6 | 35 | 22.4 |
| 18 | PD | 2.27 | 0.71 | 3.21 | 11 | 24 | 25 | 17 |
| 21 | MR | 1.96 | 1.39 | 1.41 | 27 | 28 | 53 | 39 |
| 22 | PR | 1.92 | 0.49 | 3.88 | 37.6 | 57.9 | 72.1 | 28.6 |
| 25 | NC | 1.59 | 1.00 | 1.59 | 22 | 14 | 35 | 14 |
| 26 | MR | 1.38 | 0.95 | 1.44 | 8 | 22 | 11 | 21 |

*positive in the biomarker of AST elevation referred to meeting both of ΔAST > 1.35 and ΔAST/ΔALT > 1.35

TABLE 4
RRMM Patients Negative in the Biomarker of AST Elevation
and the Efficacy after CPT Monotherapy*

| | | | | | | | After | |
|---|---|---|---|---|---|---|---|---|
| | | | | | Before | | two days | |
| | | The fold increased | | | CPT | | of CPT | |
| | Effic- | after CPT treatment | | | treatment | | treatment | |
| No | acy | ΔAST | ΔALT | ΔAST/ΔALT | AST | ALT | AST | ALT |
| 10 | NC | 6.50 | 7.95 | 0.82 | 28 | 21 | 182 | 167 |
| 13 | PD | 4.32 | 3.27 | 1.32 | 31 | 15 | 134 | 49 |
| 14 | PD | 3.50 | 2.67 | 1.31 | 8 | 21 | 28 | 56 |
| 16 | NC | 2.64 | 6.26 | 0.42 | 33 | 19 | 87 | 119 |
| 19 | NC | 2.11 | 2.20 | 0.96 | 9 | 5 | 19 | 11 |
| 20 | NC | 2.07 | 2.00 | 1.04 | 14 | 25 | 29 | 50 |
| 23 | MR | 1.79 | 2.00 | 0.89 | 14 | 11 | 25 | 22 |
| 24 | NC | 1.63 | 2.13 | 0.77 | 51 | 64 | 83 | 136 |
| 27 | MR | 1.38 | 1.50 | 0.92 | 8 | 8 | 11 | 12 |
| 28 | PD | 1.36 | 1.45 | 0.93 | 7.8 | 6.6 | 10.6 | 9.6 |
| 29 | NC | 1.24 | 1.58 | 0.78 | 21 | 24 | 26 | 38 |
| 30 | MR | 1.15 | 0.72 | 1.60 | 13 | 18 | 15 | 13 |
| 31 | NC | 1.12 | 1.25 | 0.89 | 17 | 8 | 19 | 10 |
| 32 | MR | 1.11 | 1.05 | 1.06 | 31.8 | 19.5 | 35.4 | 20.4 |
| 33 | PD | 1.06 | 0.76 | 1.40 | 21.7 | 15.2 | 23 | 11.5 |
| 34 | MR | 1.06 | 1.03 | 1.03 | 17 | 33 | 18 | 34 |
| 35 | NC | 0.94 | 0.91 | 1.04 | 34 | 11 | 32 | 10 |
| 36 | PD | 0.89 | 0.45 | 1.98 | 18 | 29 | 16 | 13 |
| 37 | PR | 0.53 | 0.60 | 0.88 | 24.7 | 20.5 | 13 | 12.2 |

*negative in the biomarker of AST elevation referred to not meeting both of ΔAST > 1.35 and ΔAST/ΔALT > 1.35

TABLE 5
Analysis on the Relevance of Clinical Response (PR + CR)
of MM Patients to CPT Treatment with the Elevation of Serum AST

| | Having | No | | |
|---|---|---|---|---|
| ΔAST > 1.35 | clinical | clinical | | |
| and ΔAST/ | response | response | OR | P |
| ΔALT > 1.35 | (n = 13) | (n = 24) | (95% CI) | value |
| Negative | 1 (7.7%) | 18 (75%) | | |
| Positive | 12 (92.3%) | 6 (25%) | 36.00 (3.84-337.98) | 0.002 |

TABLE 6
Analysis on the Relevance of Clinical Response (PR + CR)
of MM Patients to CPT Treatment with the Elevation of Serum LDH

| | Having clinical | No clinical | | |
|---|---|---|---|---|
| | response | response | OR | P |
| [symbol] LDH | (n = 6) | (n = 13) | (95% CI) | value |
| <1.75 | 1 (16.67%) | 11(84.62%) | | |
| ≧1.75 | 5 (83.33%) | 2(15.38%) | 27.50(2.0-378.84) | 0.013 |

8. Characteristics of the Changes in Serum AST and LDH After CPT Treatment

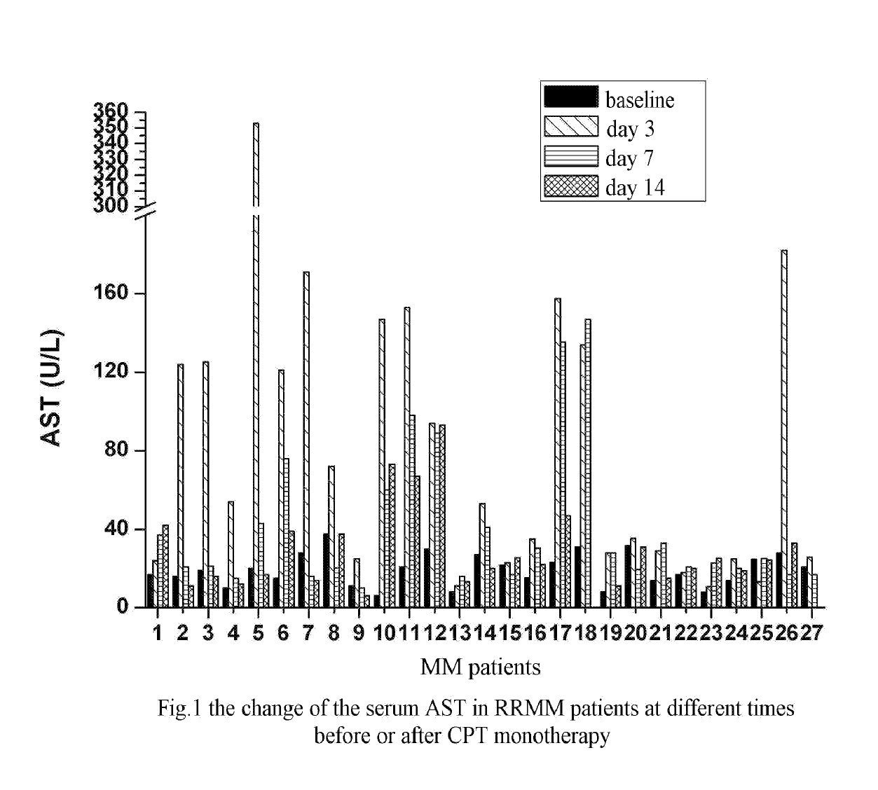

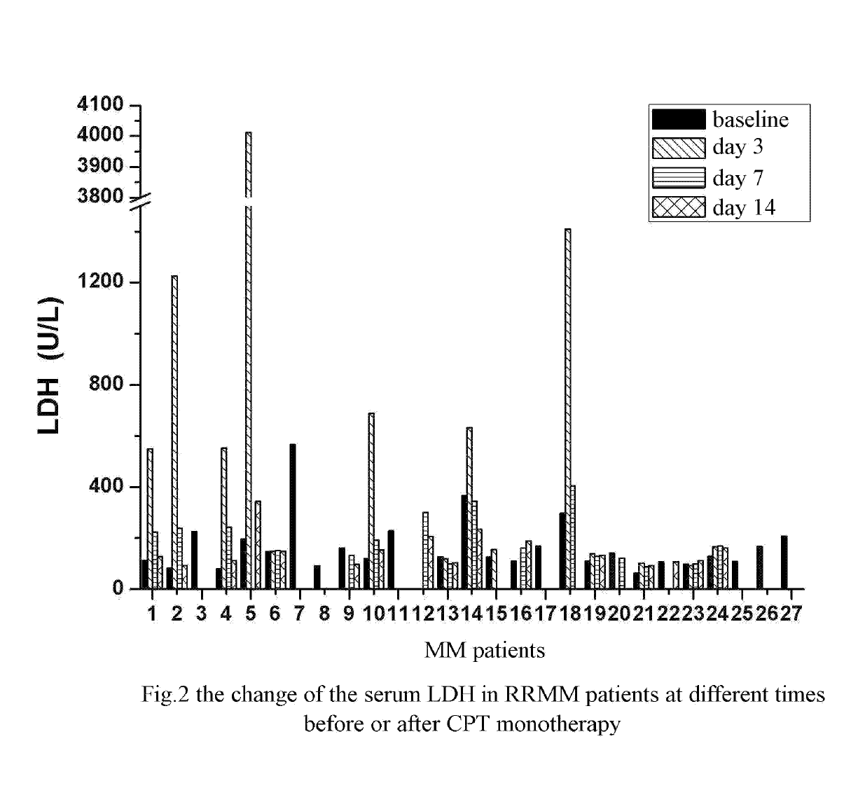

During the 14 days of continuous CPT treatment, serum AST and LDH at baseline (before the first CPT administration) and serum AST and LDH in fasting venous blood before CPT administration on day 3, day 7 and day 14 were tested respectively. It was discovered that the abnormal rise of AST or LDH had similar features, i.e., achieving a peak value on day 3, obviously falling on day 7, and reduction to normal level on day 14 for most patients (FIGS. 1 and 2). If the continuous liver damage caused by CPT occurred, the fall after the abnormal rise of serum AST or LDH was not obvious or even absent.

### EXAMPLE 2

### The Rise of Serum AST and LDH Could Predict the Efficacy of CPT in Combination with Thalidomide in the Treatment of MM Patients

1. Inclusion Criteria

(1) meeting the diagnostic criteria for MM;

(2) patient conditions: MM patients having a relapse after at least first-line chemotherapy protocol of two treatment cycles or MM patients having progress or being ineffective after the latest treatment (at least two treatment cycles) and at least the latest treatment protocol (within three months) for the patient comprised Thalidomide (abbreviated as Thal) or Thalidomide was used to retain the treatment (the usage amount of Thalidomide is no less than 100 mg/d);

(3) age≧18;

(4) physical condition grade≧60;

(5) expected lifetime≧3 months;

(6) neither chemotherapy nor radiotherapy was received within four weeks except Thalidomide, and washout period was over;

(7) no obvious dysfunctions of main organs (it was judged according to the upper limit of grade I toxicity in addition to the following indexes, see appendix 3). The following laboratory indexes must meet the following requirements:

Blood: white blood cell≧3.0×10⁹/L, neutrophilic granulocyte≧1.0×10⁹/L, platelet count≧30×10⁹/L and hemoglobin≧60 g/L.

Liver function: ALT/AST and serum total bilirubin should be within a normal range.

Renal function: creatinine clearance rate≧30 ml/min

(8) Patients signed an informed consent after understanding the details of the experiments.

2. Exclusion Criteria

Any patients meeting any one of the following criterions shall be excluded:

(1) non-secretory MM patients (there were no measurable M protein, free light chain);

(2) gestational or lactating women, and patients at reproductive age who were unwilling to take contraception measures;

(3) patients having a history of being allergic to biological products such as protein or Thalidomide or allergic constitution;

(4) patients having a history of viral hepatitis or other liver diseases, for example, liver cirrhosis, alcoholic liver disease, drug hepatitis, etc; patients positive for hepatitis B virus e antigen, surface antigen; patients positive for HBV-DNA or HCV-DNA.

(5) patients having mental diseases or a history of mental diseases;

(6) patients having severe or uncontrollable diseases of important organs (heart and cerebral vessels, respiratory, digestion, nerve, etc) within 12 months before enrollment, for example, myocardial infarction, III-IV stage of heart failure, angina pectoris, heart diseases with an obvious clinical manifestation, left ventricular ejection fraction<0.5; severe conduction dysfunction; hypotension (Sitting systolic blood pressure≦90 mmHg or Sitting diastolic blood pressure≦60 mmHg);

(7) patients having a history of deep venous thrombosis or pulmonary embolism within half a year before enrollment, patients having active bleeding or new thrombosis, patients taking anticoagulants or having a history of bleeding tendency;

(8) patients having a history of other tumors (except those having a history of basal cell carcinoma or squamous cell carcinoma of skin which already achieved CR, uterine cervix or breast adenocarcinoma in situ) within five years;

(9) those who were thought to be not suitable to be participated in this experiment;

3. Efficacy Evaluation Criteria

See Example 1.

4. Dosage and Administration Method

Three dosage groups were set for CPT treatment, 5 mg/kg, 8 mg/kg and 10 mg/kg respectively; and a single dosage of 100 mg/d was used for thalidomide (Thal). CPT was added to a 5% glucose injection of 500 ml, and the intravenous infusion was continued for 2.0 hours±15 minutes (for those who were suffering from diabetes or having a history of diabetes, normal saline of 500 ml may be used instead), once a day for 5 continuous days followed by a treatment-free period of 12±3 days as one treatment observation period (one treatment cycle). Thalidomide was orally administrated at 100 mg/day before sleep.

5. Efficacy Evaluation

A total of 29 subjects were enrolled in the study and received at least two cycles of CPT+Thal treatment. The efficacy evaluation was shown in Table 7.

TABLE 7
Efficacy of CPT + Thal for the treatment of Patients with RRMM

| Efficacy | Number of | Incidence |
|---|---|---|
| Evaluation | patients | rate (%) |
| CR | 2 | 6.8 |
| PR | 6 | 20.7 |
| MR | 3 | 10.3 |
| NC | 15 | 51.7 |
| PD | 3 | 10.3 |

6. The Relationship Between the Changes of Serum AST and LDH After CPT+Thal Treatment and the Efficacy

ΔAST was used to refer to the degree of the serum AST elevation after CPT treatment (ΔAST=serum AST after treatment/baseline level). ΔALT was used to refer to the degree of the serum ALT elevation after CPT treatment (ΔALT=serum ALT after treatment/baseline level). As the biomarkers of tumor-damage, ΔAST>1.35 and ΔAST/ΔALT>1.35 were regarded as positive in the present study, otherwise, negative. The response of the multiple myeloma patients to CPT+Thal was predicted by the change of ΔAST and ΔAST/ΔALT after CPT administration.

Table 8 showed the concentration of AST, ALT and LDH in the fasting venous blood samples collected from 29 patients after one dosing of CPT+Thal and before the second CPT+Thal dosing, and their clinical responses after having finished at least two cycles of treatment. Of 29 subjects, 12 had ΔAST>1.35 and positive for ΔAST/ΔALT tumor-damaging biomarker, with 5 achieved a PR (41.7%), 2 CR (16.7%), 3 MR (25%), 2 NC (16.6%), and the clinical response rate (PR+CR) was 58.3%. Seventeen subjects had ΔAST1.35 and negative for ΔAST/ΔALT tumor-damaging biomarker, with 1 achieved a PR (5.9%), 13 NC (76.5%), 3 PD (17.6%), and the clinical response rate (PR+CR) was 5.9% (no CR). In the patients whose tumor-damaging biomarkers were positive, the percentage of PR+CR were 9.9 times of that of patients whose tumor-damaging biomarkers were negative.

The sensitivity of this tumor-damaging biomarker in predicting the clinical response (PR+CR) of MM patients to CPT+Thal was 87.5% (7/8), and the specificity thereof was 76.19% (16/21). The predicted value of the positivity of the clinical response (PR+CR) was 58.3% (7/12) and the predicted value of the negativity of the clinical response (PR+CR) was 94.1% (16/17). A logistic regression analysis was conducted with SPSS statistics software on the relevance of clinical response rate of the MM patients to CPT+Thal with the rise of serum AST, and the result showed that ΔAST was useful for predicting whether the patients would response to CPT+Thal (P<0.01), with an odds ratio of 22.40 and 95% confidence interval of 2.19˜228.73 (Table 9).

7. The Relationship Between the Change of LDH After the Treatment of CPT+Thal and the Efficacy

After one dosing of CPT+Thal and before the second dosing, venous blood samples were collected and serum LDH levels were tested. It can be seen that serum LDH of the treated patients increased to varying degrees (Table 8). ΔLDH was used to refer to the degree of the serum LDH elevation after the treatment of CPT+Thal (ΔLDH=serum LDH after treatment/baseline level). Twenty-one subjects had a ΔLDH ratio (Table 6). ΔLDH 1.75 was regarded as positive in tumor-damaging biomarker, otherwise, negative. The response of the multiple myeloma patients to CPT+Thal was predicted by the change of ΔLDH after the administration of CPT+Thal.

There were 6 patients with positive tumor-damaging biomarker, wherein 5 of them had a clinical response of PR or better (PR+CR) (83.3%). There were 15 patients with negative tumor-damaging biomarker, wherein 2 of them had a clinical response of PR or better (PR+CR) (13.3%). In the patients whose tumor-damaging biomarker was positive, the percentage of PR+CR was obviously larger than that of patients whose tumor-damaging biomarker was negative.

The sensitivity of the tumor-damaging biomarker ΔLDH in predicting the clinical response (PR+CR) of MM patients to CPT+Thal was 71.43% (5/7), and the specificity thereof was 92.86% (13/14). The predicted value of the positivity of the clinical response (PR+CR) was 83.33% (5/6) and the predicted value of the negativity of the clinical response (PR+CR) was 86.67% (13/15). A logistic regression analysis was conducted with SPSS statistics software on the relevance of clinical response rate of the MM patients to CPT+Thal with the rise of serum LDH, and the result showed that ΔLDH was useful for predicting whether the patients would response to CPT+Thal (P<0.01), with an odds ratio of 32.50 and 95% confidence interval of 2.38 443.15 (Table 10).

TABLE 8
Changes of Serum AST, ALT and LDH and the Efficacy of RRMM
Patients after CPT + Thal Treatment

| | | | | | | Before | | | After one day | | |
|---|---|---|---|---|---|---|---|---|---|---|---|
| | | The fold increased after CPT treatment | | | | CPT treatment | | | of CPT treatment | | |
| No. | Efficacy | ΔAST | ΔALT | ΔLDH | ΔAST/ΔALT | AST | ALT | LDH | AST | ALT | LDH |
| 1 | CR | 21.67 | 2.08 | 19.10 | 10.40 | 24 | 12 | 97 | 520 | 25 | 1853 |
| 2 | PR | 10.50 | 1.50 | | 7.00 | 28 | 8 | 971 | 294 | 12 | ND |
| 3 | CR | 9.60 | 1.62 | 6.74 | 5.93 | 25 | 21 | 215 | 240 | 34 | 1449 |
| 4 | PR | 5.58 | 0.80 | 3.38 | 6.97 | 19 | 25 | 143 | 106 | 20 | 483 |
| 5 | NC | 4.76 | 6.00 | | 0.79 | 17 | 5 | 64 | 81 | 30 | ND |
| 6 | PR | 4.76 | 0.85 | 5.10 | 5.57 | 29 | 37.2 | 164 | 138.1 | 31.8 | 836 |
| 7 | PR | 4.28 | 0.86 | 6.44 | 4.95 | 30.6 | 28 | 171 | 131 | 24.2 | 1101 |
| 8 | MR | 3.56 | 1.30 | | 2.75 | 52.2 | 40.5 | ND | 185.9 | 52.5 | 2060 |
| 9 | MR | 3.46 | 0.73 | 2.31 | 4.76 | 13 | 11 | 117 | 45 | 8 | 270 |
| 10 | MR | 3.20 | 0.97 | | 3.29 | 30 | 36 | ND | 96 | 35 | ND |
| 11 | NC | 2.65 | 1.05 | | 2.52 | 23 | 19 | ND | 61 | 20 | ND |
| 12 | NC | 2.38 | 2.71 | | 0.88 | 32 | 38 | ND | 76 | 103 | ND |
| 13 | PR | 1.73 | 1.23 | 1.40 | 1.40 | 11 | 13 | 121 | 19 | 16 | 170 |
| 14 | NC | 1.73 | 1.25 | | 1.38 | 11 | 8 | 172 | 19 | 10 | ND |
| 15 | NC | 1.30 | 0.60 | 0.96 | 2.16 | 16.7 | 17.6 | 162 | 21.7 | 10.6 | 156 |
| 16 | PD | 1.21 | 1.00 | 0.88 | 1.21 | 14 | 12 | 149 | 17 | 12 | 131 |
| 17 | PR | 1.19 | 1.03 | 1.54 | 1.15 | 15.6 | 9.5 | 120 | 18.5 | 9.8 | 185 |
| 18 | NC | 1.11 | 0.78 | 1.16 | 1.42 | 19 | 18 | 182 | 21 | 14 | 211 |
| 19 | PD | 1.09 | 0.42 | 0.85 | 2.58 | 18.2 | 23.2 | 131 | 19.8 | 9.8 | 111 |
| 20 | NC | 1.06 | 1.20 | 1.18 | 0.88 | 17 | 10 | 95 | 18 | 12 | 112 |
| 21 | NC | 1.00 | 0.85 | 0.95 | 1.18 | 13 | 13 | 131 | 13 | 11 | 125 |
| 22 | NC | 0.97 | | 0.98 | | 34 | 41 | 189 | 33 | ND | 185 |
| 23 | NC | 0.91 | 0.81 | 0.71 | 1.12 | 11 | 16 | 142 | 10 | 13 | 101 |
| 24 | NC | 0.89 | 0.75 | 0.78 | 1.19 | 19 | 12 | 135 | 17 | 9 | 105 |
| 25 | NC | 0.89 | 0.71 | | 1.24 | 9 | 7 | 101 | 8 | 5 | ND |
| 26 | NC | 0.88 | 0.75 | 0.82 | 1.18 | 17 | 16 | 168 | 15 | 12 | 138 |
| 27 | PD | 0.80 | 0.83 | 0.90 | 0.96 | 15 | 6 | 145 | 12 | 5 | 131 |
| 28 | NC | 0.58 | 1.02 | 0.55 | 0.56 | 33 | 51 | 334 | 19 | 52 | 183 |
| 29 | NC | 0.52 | 0.44 | 0.69 | 1.19 | 25 | 55 | 167 | 13 | 24 | 115 |

TABLE 9
Analysis on the Relevance of Clinical Response
of the Multiple Myeloma Patients to the treatment
of CPT + Thal with the Rise of Serum AST

| | Having | No | | |
|---|---|---|---|---|
| ΔAST > 1.35 | clinical | clinical | | |
| and ΔAST/ | response | response | OR | P |
| ΔALT > 1.35 | (n = 13) | (n = 24) | (95% CI) | value |
| Negative | 1 (12.5%) | 16 (76.19%) | | |
| Positive | 7 (87.5%) | 5 (23.81%) | 22.40 (2.19~228.73) | 0.009 |

TABLE 10
Analysis on the Relevance of Clinical Response (PR +
CR) of the Multiple Myeloma Patients to the treatment
of CPT + Thal with the Rise of Serum LDH

| | Having clinical | No clinical | | |
|---|---|---|---|---|
| | response | response | OR | P |
| [symbol] LDH | (n = 7) | (n = 14) | (95% CI) | value |
| <1.75 | 2 (28.57) | 13(92.31%) | | |
| ≧1.75 | 5 (71.43%) | 1(7.69%) | 32.50 (2.38~443.15) | 0.009 |

1. A method for early evaluation of clinical efficacy of an antitumor intervention measure, comprising:
evaluating the efficacy of the antitumor intervention measure by assaying whether the content of a tumor-damaging biomarker(s) in the blood of a patient having tumor rises as compared to the baseline level before treatment within a time window after the patients receives at least one antitumor intervention measure.

2. The method of claim 1, wherein said tumor is hematological tumor.

3. The method of claim 2, wherein said hematological tumor is selected from a group consisting of myelodysplastic/myeloproliferative disease (MDS/MPD), myelodysplastic syndrome (MDS), leukemia, B cell tumor, T/NK cell tumor, hodgkin lymphoma.

4. The method of claim 3, wherein said mature B cell tumor is selected from a group consisting of chronic lymphocytic leukemia/small lymphocytic lymphoma, prolymphocytic leukemia, lymphoplasmacytic lymphoma/macroglobulinemia, splenic marginal zone B-cell lymphoma, splenic marginal zone B-cell lymphoma with villous lymphocyte, hairy cell leukemia B cell tumor, plasmacytoma (comprising multiple myeloma), MALT extranodal (nodal) marginal zone B cell lymphoma, follicular lymphoma, mantle cell lymphoma, diffuse large B cell lymphoma, Burkitt's lymphoma, lymphomatoid granulomatosis, wherein plasma cell myeloma/plasmacytoma is preferable.

5. The method of claim 1, wherein said antitumor intervention measure is selected from a group consisting of chemotherapy and biologically targeted therapy.

6. The method of claim 1, wherein said antitumor intervention measure is single administration or combined administration.

7. The method of claim 1, wherein said antitumor intervention measure is an intervention measure resulting in death of tumor cells, wherein said intervention measure resulting in death of tumor cells is selected from a group consisting of cytotoxic drugs and targeted drugs inducing apoptosis of tumor cells.

8. The method of claim 7, wherein said targeted drugs inducing apoptosis of tumor cells are antitumor drugs acting on CD20 antigen, epidermal growth factor receptor (EGFR), tyrosine kinase, proapoptotic receptor, and proteasome.

9. The method of claim 1, wherein said tumor-damaging biomarker(s) is selected from a group consisting of Alanine Aminotransferase (ALT), Aspartate Transaminase (AST), Lactate Dehydrogenase (LDH), Uric acid, Creatinine, Monoclonal Immunoglobulin (or M protein), Immunoglobulin (IgG, IgA, IgD, IgM, IgE), Free Light Chain (FLC), β2 microglobulin (β2-MG).

10. The method of claim 1, wherein the time window for assaying said tumor-damaging biomarker(s) is 8 to 48 hours after receiving one treatment of antitumor intervention measure.

11. The method of claim 1, wherein the rise of the tumor-damaging biomarker(s) refers to 10% or more above the baseline value.

12. The method of claim 1, wherein the tumor-damaging biomarker(s) is selected from a group consisting of Alanine Aminotransferase (ALT), Aspartate Transaminase (AST), Lactate Dehydrogenase (LDH); said tumor is multiple myeloma; said antitumor intervention measure is the administration of CPT alone or the administration of CPT in combination with thalidomide.

13. Use of reagents assaying a tumor-damaging biomarker(s) in producing a reagent or kit for early evaluation of clinical efficacy of an antitumor intervention measure, wherein said early evaluation of clinical efficacy of the antitumor intervention measure comprises:
evaluating the efficacy of the antitumor intervention measure by assaying whether the content of a tumor-damaging biomarker(s) in the blood of a patient having tumor rises as compared to the baseline level before treatment using the reagents assaying the tumor-damaging biomarker(s) within a time window after the patients receives at least one antitumor intervention measure.

14. The use of claim 13, wherein the tumor-damaging biomarker(s) is selected from a group consisting of Alanine Aminotransferase (ALT), Aspartate Transaminase (AST), Lactate Dehydrogenase (LDH).

15. The method of claim 7, wherein the proapoptotic receptor agonists are selected from the group consisting of TRAIL/APO2L or CPT, a mutant of TRAIL/APO2L, and death receptor agonists.

16. The method of claim 1, wherein the rise of the tumor-damaging biomarker(s) refers to 20% or more above the baseline value.

17. The method of claim 1, wherein the rise of the tumor-damaging biomarker(s) refers to 30% or more above the baseline value.
